(19) United States
(12) Patent Application Publication
(10) Pub. No.: US 2006/0165783 A1
(43) Pub. Date: Jul. 27, 2006

(54) Compacted lactic acid menthyl ester

(76) Inventor: Alfred Korber, Holzminden (DE)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/608,416

(22) Filed: Jun. 30, 2003

(30) Foreign Application Priority Data
Jul. 1, 2002 (DE) 102 29 472.0

(51) Int. Cl. A61K 9/20; A61K 31/22

(52) U.S. Cl. 424/464; 514/546

(57) ABSTRACT

The invention relates to lactic acid menthyl ester compacts, a method for the preparation of lactic acid menthyl ester compacts and the use thereof.

The invention relates to lactic acid menthyl ester compacts, a method for the preparation of lactic acid menthyl ester compacts and the use thereof.

Lactic ester menthyl ester (LME, menthyl lactate) of the formula (1) is a compound that has a physiological cooling action on skin and mucous membranes of the body and can be used in a multiplicity of products where a long-lasting physiological cooling effect is desired, such as, for example, in luxury goods such as chewing gums, chewing tobacco, cigarettes, ice cream, confectionery and drinks, as well as in toiletries and pharmaceutical or cosmetic preparations such as dentifrices, mouthwashes, perfumes, powders, lotions, ointments, oils, creams, sun screen preparations, shaving creams and aftershaves, shower gels or shampoos. Advantageously 1-lactic acid 1-menthyl ester (1b) is used in these products.

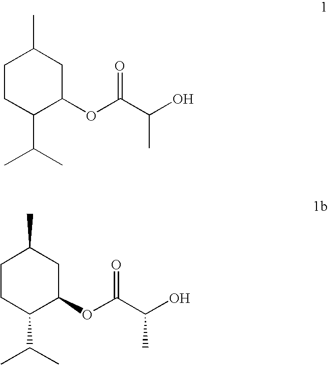

LME is commercially available in the form of solidified distillate or also in the form of the crystalline product. On storing for a prolonged period, usually in the course of several weeks, LME develops an acidic, pungent odor, as a result of which it becomes unusable for the majority of intended applications. Moreover, the solid product often has to be melted several times before metering and incorporation in products or articles. This thermal loading additionally lowers the quality of the LME. This loss in quality is frequently accompanied by a rise in the acid number.

LME is usually prepared by acid-catalyzed etherification of commercially available lactic acid with commercially available menthol, as, for example, described in EP-B 794 169. For further purification the distillate can be subjected to crystallization; (1), which is obtainable by this means, usually has a purity of greater than 99.5% (GC).

To prevent the acidic, pungent note, a stabilizer in the form of alkali metal and/or alkaline earth metal carbonates and/or bicarbonates can be added during the crystallization process, as described in EP-B 794 169. To achieve the desired stabilization, in this case the crystallization is carried out in the presence of the said inorganic salts. The mixture of LME and inorganic salt obtainable by this method is, however, disadvantageous for some applications, especially if the inorganic salt gives rise to incompatibilities, turbidities or precipitates when the mixture is used.

The aim of the present invention was, now, to achieve stabilization of lactic acid menthyl ester without the addition of inorganic salts.

It has now been found that molded products (compacts) formed by pressing (compacting) the lactic acid menthyl ester are stable for a long storage period (at least 6 months) and do not change from the sensory standpoint. The acid number of this pressed lactic acid menthyl ester does not change during this storage period.

The subject of the present invention is therefore compacts having a lactic acid menthyl ester content of at least 80% (m/m).

A further subject of the present invention is a method for the preparation of lactic acid menthyl ester compacts, wherein the lactic acid menthyl ester content in the finished compact is at least 80% (m/m).

An LME content in the compact of at least 90% (m/m) is preferred, of at least 95% (m/m) is particularly preferred and of at least 98% (m/m) is very particularly preferred. In a preferred embodiment the LME content in the compact is more than 99% (m/m). The various degrees of purity can, for example, be obtained by distillation or crystallization.

According to the invention all isomers of lactic acid menthyl ester can be used in the compression, i.e. in principle the lactic acid esters of menthol, neomenthol, isomenthol and/or neoisomenthol can be used. These LMEs can be used in the compression as individual isomers or also as arbitrary mixtures. In this context the menthyl ester can be the menthyl ester of d-lactic acid, of 1-lactic acid or of an arbitrary mixture thereof, such as, for example, of the racemic d1-lactic acid. Advantageous lactic acid esters are those of menthol, preferably of 1-menthol. A very particularly preferred LME is 1-lactic acid 1-menthyl ester (1b).

The LME is preferably introduced into the compression in solid form, for example in the form of crystals, flakes, granules, powders, or mixtures of these forms. Flakes of LME are preferred according to the invention. Flaking of the LME can, for example, be carried out analogously to the method described in U.S. Pat. No. 3,064,311, in which a liquid LME is made into flakes via a cooled flaking roller.

According to the invention the LME compacts do not contain any alkali metal and/or alkaline earth metal carbonates and/or bicarbonates as stabilizers. An addition of other substances having a stabilizing action is also not necessary.

The compacts can be used as such in the applications, as a result of which thermal loading as a result of melting is avoided. Furthermore, convenient and reliable handling of the compacts when incorporating in formulations, preparations, articles and products is provided. The compacts have good pourability and flowability, which is advantageous for metering, especially in the engineering and industrial field.

The compacts can have various shapes. The most common shapes are spheres, cubes, cuboids, cushions, cylinders, tablets, pellets or briquettes; the pellet is a preferred shape.

The compacts according to the invention formed by compacting remain dimensionally stable for several months on storage. Agglomeration, caking or intergrowth of the compacts does not take place. The compacts according to the invention therefore have good pourability and flowability, as well as meterability, even after prolonged storage times.

The dimensions of the compact can differ substantially. In the case of a sphere the diameter is usually in the range 2-20 mm, preferably 4-10 mm. In the case of the pellet, length and width are usually 3-20 mm, preferably 5-15 mm and the height usually in the range of 2-15 mm, preferably 5-12 mm.

The pressing force of the compactor is usually in the range of 10-100 kN (kilo-newton), preferably 30-80 kN.

Linear pressing forces of 1.5-4 newton/mm.mm, based on the diameter, are preferably to be used when compacting the lactic acid menthyl ester.

Briquetting methods which have proved their worth are described, for example, in Chemie−Anlagen+Verfahren 1985, No. 4, pages 51, 54 and 59.

The following example illustrates the invention.

### EXAMPLE

150-200 kg flaked lactic acid menthyl ester (CC content: 97.3% 1-lactic acid 1-menthyl ester, 1.4% d-lactic acid 1-menthyl ester) per hour are fed via a screw with downstream vibratory chute to a compactor from BEPEX GmbH consisting of two compactor rollers (dimensions of the compactor rollers: width 100 mm, diameter 200 mm). At room temperature, using a pressing force of 50 kN the compactor produced compacts in the form of pellets, cushions or cylinders with dimensions of length=width=10 mm and height=6 mm.

What is claimed is:

1. Lactic acid menthyl ester compacts, wherein the lactic acid menthyl ester content in the compact is at least 80% (m/m).

2. The compacts as claimed in claim 1, wherein the lactic acid menthyl ester content in the compact is at least 90% (m/m).

3. The compacts as claimed in claim 1, wherein the lactic acid menthyl ester content in the compact is at least 95% (m/m).

4. The compacts as claimed in claim 1, wherein said lactic acid menthyl ester is 1-lactic acid 1-menthyl ester.

5. The compacts as claimed in claim 1, wherein said compact is formed by a process comprising compression of ingredients and flaked lactic acid menthyl ester is used in said compression.

6. The compacts as claimed in claim 1 exhibiting the form of spheres, cubes, cuboids, cushions, cylinders, tablets, pellets or briquettes.

7. A method for the preparation of lactic acid menthyl ester compacts according to claim 1, wherein the lactic acid menthyl ester content in the finished compact is at least 80% (m/m).

8. The method as claimed in claim 7, wherein the lactic acid menthyl ester content in the finished compact is at least 90% (mm).

9. The method as claimed in claim 7, wherein said compact is formed by a process comprising compression of ingredients and flaked lactic acid menthyl ester is used in said compression.

10. The method as claimed in claim 7, wherein said lactic acid menthyl ester is 1-lactic acid 1-menthyl ester is used.

11. The method as claimed in claim 7, wherein said compact is formed by a process comprising compression of ingredients and the compression is carried out using a pressing force in the range of 10-100 kN.

12. The use of compacts as claimed in claim 1 for incorporation in products where a long-lasting physiological cooling effect is desired on skin and mucous membranes.
